(19) United States
(12) Patent Application Publication
(10) Pub. No.: US 2005/0130158 A1
(43) Pub. Date: Jun. 16, 2005

(54) Screening method

(76) Inventors: Jeremy Tavare, Bristol (GB); Bruno Marks, Bristol (GB); Laura Fletcher, Bristol (GB)

Correspondence Address: WELSH & KATZ, LTD, 120 S RIVERSIDE PLAZA, 22ND FLOOR, CHICAGO, IL, 60606, US

(21) Appl. No.: 10/502,770

(22) PCT Filed: Feb. 18, 2003
(86) PCT No.: PCT/GB03/00707

(30) Foreign Application Priority Data
Feb. 19, 2002 (GB) 0203924.6

(51) Int. Cl. C12Q 1/68; C12N 15/85

(52) U.S. Cl. 435/6; 435/455

(57) ABSTRACT

There is disclosed an assay to determine the effect of an applied agent or influence or other stimulus on the sub-cellular localisation of the expressed product of a nucleotide sequence (“the nucleotide sequence under investigation”), comprising: (i) transfecting a cell population with a cloned expression vector comprising a composite nucleotide sequence encoding the nucleotide sequence under investigation and a detectable tag, arranged such that the product of the expressed composite nucleotide sequence includes the expressed detectable tag; (ii) applying the applied agent, influence or other stimulus to at least a portion of the transfected cell population; (iii) observing the sub-cellular location of the detectable tag in the transfected cell population and comparing the observed sub-cellular localisation to the sub-cellular localisation of the detectable tag in the cell population in the absence of said applied agent or influence.

### FIELD OF THE INVENTION

The present invention relates in one aspect to an assay to determine the effect of a stimulus, such as an applied agent or influence, on the sub-cellular localisation of the expressed product of a nucleotide sequence which has been introduced into the cell. Information obtained using this assay may be useful in establishing the function of the nucleotide sequence. Where the stimulus has such an effect, this may be used as the basis for screening chemical compounds to identify “hit” compounds which alter the response of the expressed product of the nucleotide sequence to the stimulus. The assays of the invention may be carried out in parallel and at high throughput.

### BACKGROUND OF THE INVENTION

The elucidation of the human genome sequence provides a comprehensive collection of human sequences for use in the identification of many thousands of potential new drug targets. However, to identify these targets, a function must first be assigned to each of the 30,000 or more genes encoded by the human genome. Many approaches have been proposed to assign functions to gene sequences including bioinformatics, structural genomics, gene arrays and proteomics.

Many pharmaceutical and biotechnology companies are making increasing use of cell-based screening systems for drug discovery. Receptor-agonist interactions, intracellular Ca²⁺ measurements, and gene expression using fluorescent or luminescent reporters can be performed using standard microtitre plate readers such as the Cytofluor, LJL Analyst or FLIPR. All such systems rely on the measurement of changes occurring within populations of cells.

Currently, the most common way to perform a compound screen is to take a purified target protein and measure its activity or function in 96 or 384 well plates in the presence or absence of the compounds (i.e. an in vitro screen). Such screens can be performed at ultra-high throughput (uHTS), often allowing well in excess of 100,000 compounds to be evaluated in a single day. Compounds which exhibit activity in such a primary screen are then examined further for their effects on intact cell systems. Unfortunately, compounds identified by in vitro screening are often later found (i) not to get into living cells, (ii) to be inactive in the presence of the complex plethora of other proteins found inside cells, or (iii) to have non-specific toxic side effects. This problem can generate a substantial time delay in drug discovery and wastes valuable resources.

### SUMMARY OF THE INVENTION

In a first aspect, the present invention provides an assay in which functional information pertaining to the expressed product of a nucleotide sequence can be determined. This assay is also referred to herein as the “primary assay”. Specifically, in this assay, a cell population which has been transfected with a cloned expression vector comprising the nucleotide sequence and a sequence encoding a detectable tag is subjected to a known agent or influence or other stimulus, and the sub-cellular location of the detectable tag (and hence the expressed product of the nucleotide sequence) determined and compared with the sub-cellular location of the detectable tag in the absence of the applied agent or influence. Where the applied agent or influence has had an effect on the sub-cellular location of the expressed nucleotide sequence (as indicated by the detectable tag), this provides an indication of function of the expressed nucleotide sequence.

The expression “a known agent or influence or other stimulus” used herein refers particularly to an agent, influence or other stimulus which is deliberately applied to the cell population. Normally, the application of the agent, influence or other stimulus will be performed in a reasonable expectation of eliciting some form of response from the cell, and the amount of the agent, influence or other stimulus applied, as well as the general conditions of its application, will be selected accordingly. The response may be well defined from a scientific point of view, or may be relatively poorly defined. The response mechanism may be well understood from a scientific point of view, or may be relatively poorly understood. Generally speaking, the better the scientific definition of the response, and the better the scientific understanding of the response mechanism, the more functional information pertaining to the expressed product of the nucleotide sequence can be obtained from the assay. This is because that expressed product can be identified as potentially associated with relatively more, or relatively more detailed, or relatively better understood, aspects of cellular function or dysfunction, for example one or more aspects of cellular function or dysfunction relevant to human, animal or plant health.

For example, where insulin is used as the applied agent and this has an effect on the sub-cellular location of the expressed nucleotide sequence (as indicated by the detectable tag), this provides an indication that the expressed nucleotide sequence might be involved in the insulin signalling pathway, or some aspect of the regulation of metabolism. The nucleotide sequence might therefore be considered a candidate ‘diabetogene’—a useful therapeutic screening target for the treatment of diabetes. Inevitably further characterisation of the expressed nucleotide sequence would need to be carried out to validate it as a diabetogene and good target for screening.

Thus, in summary, the assay of the first aspect of this invention is highly suitable for use in a program to elucidate the function of the expressed product of a nucleotide sequence whose function is unknown, or about which limited functional information is available.

This primary assay or screen of the invention may be repeated a plurality of times (either sequentially or simultaneously) in respect of different nucleotide sequences using the same applied agent or influence, in order to screen a large number of nucleotide sequences; the ones which code for a product whose sub-cellular location is affected in the presence of the stimulus can be identified. A clone identified in this way can be used in further assays or screens in which the effect of a series of candidate drugs (typically contained in a compound library), in the presence of the agent/influence used in the primary assay, can be investigated. Through such further assays, “hit” compounds can be identified which affect translocation or sub-cellular localisation of the expressed product of the clone in the presence of the agent/influence used in the primary assay. A “hit” compound may be further screened in so-called “secondary screens” in which the effect of the hit compound on other clones is investigated in order to determine the specificity of the hit compound.

### DETAILED DESCRIPTION OF THE INVENTION

The present invention will now be discussed in more detail, first in relation to the primary screen of the invention.

A. Primary Screen

As discussed above, the primary screen of the present invention is a screen for gene function and is carried out in relation to nucleotide sequences about which little or no functional information is available (the nucleotide sequence or sequences under investigation). The primary screen of the invention has the following basic steps:
(a) transfecting a cell population with a cloned expression vector comprising a composite nucleotide sequence encoding the nucleotide sequence under investigation and a detectable tag, arranged such that the product of the expressed composite nucleotide sequence includes the expressed detectable tag;
(b) applying a known agent or influence or other stimulus to at least a portion of the transfected cell population; and
(c) observing the sub-cellular location of the detectable tag in the transfected cell population and comparing the observed sub-cellular localisation to the sub-cellular localisation of the detectable tag in the cell population in the absence of said applied agent or influence.

Thus, in accordance with this aspect of the invention, the sub-cellular location of the detectable tag is determined in the presence and the absence of the applied agent or influence, or other stimulus. Where the applied stimulus is observed to have an effect on the sub-cellular localisation of the detectable tag (which results from its effect on the expressed product of the nucleotide sequence), this provides useful information in the task of assigning a function to the nucleotide sequence. In addition, the initial, or basal, localization of the expressed product of the nucleotide sequence can also provide information for the elucidation of gene function.

The information that can be gained using the present invention, together with prior or subsequent knowledge gained by other methods such as bioinformatics can give significant insight into the function of the product of a gene sequence. With a knowledge of function of the products of gene sequences comes the potential to target them for therapeutic intervention if their arena of function is one which is affected by any given disease or condition.

The Nucleotide Sequence

The primary screen of the first aspect of the invention may be carried out in respect of a single nucleotide sequence. However, and as discussed in more detail below, the main advantages of the invention flow from the repeated use of the primary screen (preferably in parallel operations or in batches of parallel operations) in respect of different nucleotide sequences, such as those which may be obtained from libraries of nucleotide sequences.

Although in theory the present invention could be used in respect of RNA sequences, it is chiefly intended for use in respect of DNA sequences.

The nucleotide sequence which is subjected to the primary screen of the invention may be a sequence about which little or no functional information is known. For instance it may be randomly generated or may be part of a non-specific library. Alternatively, the nucleotide sequence may be a sequence about which some functional information is known. For instance, it may be a sequence that is suspected, from homology (in nucleotide sequence, amino acid sequence or polypeptide structure), to encode a member of a certain protein family or domain family.

The nucleotide sequence (or sequences where the screen is carried out in respect of a series of different nucleotide sequences) may be a full length cDNA, or a part-length sequence encoding a protein domain. The nucleotide sequence may also be an expressed sequence tag (EST) or another sequence about which very little is known from sequence homology or other methods.

As mentioned above, the nucleotide sequence may be derived from a collection of ESTs or cDNA sequences identified on the basis of homology cloning or other biochemical-based means. An advantage of specifically defined collections of ESTs or cDNA sequences is that they allow the restriction of the number of sequences requiring screening and focus the screening procedure on the expression of candidate sequences more likely to be of interest, thereby reducing the screening time involved, when compared with the use of randomly generated or non-specific libraries. For example, the primary screen of the invention could be used in respect of a collection of ESTs known to code for specific protein domains, such as PH (pleckstrin homology) domains, SH2 domains, PDZ domains, etc., which may have been identified on the basis of, for example, homology cloning or by more biochemically-based means.

As ESTs may represent internal coding sequences and thus lack either or both start and stop codons, the EST sequences could be fused to either the N— or C-terminus of the sequence encoding the detectable tag.

Further, as the ESTs are only partial sequences, some may have become de-regulated such that they exhibit constitutively active or dominant-negative characteristics, for example, as a result of the deletion of regulatory sequences. This allows the screening of expressed sequences without the complication of active regulatory molecules influencing the observed characteristics of the expressed sequences.

The use of cDNAs in the primary screen of the present invention has the advantage that the peptide expressed from tagged fusions containing near full-length cDNA sequences will be more likely to fold correctly and target appropriately than the shorter length ESTs. Further, as these cDNAs are substantially full length, the expressed peptide is more likely to retain biological activity. However, due to the possibility of stop codons in the cDNA sequence, it is generally preferred that the cDNA sequences are not fused to the 5′ end of the sequence encoding the detectable tag, or for the stop codon to be removed if a 3′ tag is desired.

Where the nucleotide sequence comprises a domain, the domain may be obtained using genomic template DNA or cDNA and primers designed against domain-specific consensus sequences. The PCR product may then be cloned using standard techniques well known to the skilled person. Due to the nature of the domain sequences, the expressed peptides are likely to fold correctly in the cell and translocate in a manner representative of the protein of which they normally form a part.

As used herein, the term “domain” means a structurally defined compact globular portion of a protein nucleotide. Some proteins consist of a single domain, and others of two or more domains joined by less structured portions of polypeptide chain.

In embodiments of the primary screen of the present invention in which the screen is carried out in respect of a series of nucleotide sequences, the nucleotide sequences may be chosen to encode products of related function. As mentioned above, screening for the function of an unknown nucleotide sequence is more focused if the nucleotide sequences being screened are, or are suspected to be, members of a specific protein or domain family e.g. tyrosine kinases, serine/threonine phosphatases, PH domains, SH2 domains etc., as the information already known about that family may assist in the selection of primary agents or influences to be used in the primary screens.

The Detectable Tag

The assay of the present invention requires the expressed nucleotide sequence to be suitably labelled using a detectable tag. The detectable tag may be any polypeptide tag, the location of which in the cell may be detected in a suitable detection method.

Examples of detection methods that may be used, in conjunction with an appropriate tag, include: fluorescence detection using fluorescence microscopy (which may be of a wide field, confocal, 2-photon etc. type) or non-microscopy based methods such as a fluorescent plate reader. Other tags that may be used include those which give colorimetric readouts, which can be detected spectrophotometrically.

The detectable tag may, for example, be a fluorescent polypeptide, or a protein sequence readily detected by a fluorescently labelled probe (e.g. an antigenic sequence and fluorescently labeled antibody). Examples of suitable fluorescent polypeptides are GFP, EGFP, and CFP. Examples of protein sequences readily detected by a fluorescently labelled probe are haemagglutinin, myc, FLAG and GST (which specific examples work by acting as epitopes for specific, fluorescently labelled antibodies) and Avidin/Biotin.

Tags may also be detected using non-fluorescence based methods. Colorimetric readouts may be used. The tag may be detected by a probe conjugated to a coloured compound that is readily detected spectro-photometrically.

If signal amplification is required, a technique that may be used is for the tag to be detected by a probe conjugated to an enzyme. This enzyme then converts substrate molecules from a non-detectable form to a detectable form. Substrate molecules may give colorimetric or fluorescent readouts (other readouts are also possible such as deposition of electron dense compounds for detection by electron microscopy). Enzyme Linked Immunosorbant Assay (ELISA) is a commonly used method of this type. Commonly used enzyme conjugates are Horse Radish Peroxidase (HRP) and Alkaline Phosphatase, though there are many others. Substrates may include molecules such as Amplex Red (Mol. Probes) that can be detected both fluorometrically and colorimetrically. Another example of signal amplification of this type is Tyramide Signal Amplification (Mol. Probes).

Other detectable tags, and methods for their detection may be used.

Although it is rare for the fusion of detectable tags, such as GFP, to an expressed nucleotide sequence to alter the characteristics of the protein/polypeptide product of the expressed nucleotide sequence, it may in some circumstances be desirable, prior to carrying out the primary screen of the invention, to test different detectable tags in parallel on expressed nucleotide sequences in order to confirm that the tag is not contributing significantly to the biological activity displayed by the fusion product of the expressed nucleotide sequence fused to the sequence encoding the detectable tag.

Further Nucleotide Sequence Modifications

In some circumstances it may be useful to make further additions, deletions or other modifications to the nucleotide sequence in order to better detect its sub-cellular localisation or changes in its sub-cellular localisation. An example of a modification that may be made is the addition of nucleotide sequences to prevent entry of the product of an expressed nucleotide sequence into the nucleus of cells (which can commonly occur if the product of the expressed nucleotide sequence is small). This might be undesirable if the expressed nucleotide sequence would not normally reside in the nucleus. The nucleotide sequences added to prevent such nuclear accumulation might include appending multiples of the detectable tag to the expressed nucleotide sequence in order to make the product of the expressed nucleotide sequence too large to enter the nucleus. Alternatively a nuclear export sequence might be used. Nuclear export sequences have been identified in numerous proteins e.g Histone Deacetylase 6 (HDAC6), Protein Kinase Inhibitor (PKI) Adenomatous Polyposis Coli (APC), etc. These sequences all mediate the export, out of the nucleus, of the polypeptide in which they reside.

Cloning

In the primary assay of the present invention, the nucleotide sequence and detectable tag (together with any other additional nucleotide sequence, such as one coding for a nuclear export signal) may be cloned into a suitable expression vector. In some circumstances, preformed expression vectors including the detectable tag may be available commercially in which case it is only necessary to clone the nucleotide sequence into the vector. The necessary cloning procedures are very well known in the art.

Transfection

In the primary screen of the present invention, the transfected cell population is obtained by transfecting a cell population with the cloned expression vector comprising the tagged nucleotide sequence encoding the expressed tagged product.

Expression clones may be transfected into tissue culture cells. Transfection methods may include lipid mediated systems e.g. Fugene (Roche), calcium chloride mediated methods and viral expression systems e.g. AdenoX and RetroX (Clontech). Other transfection systems may be used where appropriate. The cells used may be of a wide variety but they should normally be adherent. For example the cells may be made adherent by the use of e.g. culture plate coatings such as collagens, poly-lysine, antibodies etc. Ideally the cells chosen will be easy to transfect by one of the above systems and their biological characteristics will be appropriate for the screen being carried out. Cells that have been found appropriate include HeLa, NIH 3T3, VERO, CHO and PC12; other cell types appropriate for different screens may also be used.

Once transfected, the cell population may be maintained in a suitable growth medium before application of the stimulus. Alternatively, a stable cell line may be generated from the transfected cell population, enabling storage and future use of cells expressing the nucleotide sequence of interest. Generation of a stable cell line may also be used as a means to achieve a cell population in which all cells are expressing a nucleotide sequence of interest and at similar levels of expression if that is desired. The techniques involved in the generation of a stable cell line are well known to one skilled in the art.

Applied Stimulus

In the primary screen of the present invention, a known agent or influence or other stimulus is applied to the transfected cell population or to a portion thereof. Normally, the stimulus will be applied exogenously; however, in some circumstances, the agent may be generated intracellularly, for example by co-expression within the cells of another protein, in addition to the nucleotide sequence under investigation.

When screening nucleotide sequences of entirely unsuspected function it may be preferable to test a range of ‘primary stimuli’ as it may not be possible to predict which stimuli (if any) might elicit a response. Thus, the primary screen would be run using a selection of stimuli.

On the other hand, where the nucleotide sequences being screened are all likely members of a given protein or domain family e.g. tyrosine kinases, serine/threonine phosphatases, PH domains, SH2 domains etc., the information already known about that family may aid in the selection of a suitable ‘primary stimulus’ to be used.

If it is desired to search for new genes in a specific arena of function, a limited range of ‘primary stimuli’, known to be associated with the functional arena of interest, might each be used for the screening of large numbers of gene sequences. For example if the functional arena of interest is proteins involved in transducing signals from the EGF receptor, a candidate ‘primary stimulus’ to use would be EGF or an EGF mimetic, or a compound known to stimulate the ‘upstream end’ of the signal transduction pathway from the EGF receptor.

Where a response to a ‘primary stimulus’ is obtained from the product of an expressed gene sequence using the primary assay, it may be desirable to test further, related, ‘primary stimuli’ in order to try to delineate more accurately the arena of function of the gene sequence. For example, if the product of a gene sequence functions at a point in pathways of signal transduction that can be stimulated by the activation of a number of cell surface receptors, it may be expected that a response would be obtained in screens with a range of different ‘primary stimuli’. If, on the other hand, the product of the gene sequence functions just downstream of one particular cell surface receptor, one may expect that only one or very few ‘primary stimuli’ would elicit a response. The response of an expressed gene sequence to different ‘primary stimuli’ can, in this way, be used to help delineate the functional characteristics of the expressed gene sequence, such as the signal transduction pathway in which it is involved and its approximate position on that pathway.

In addition to applied agents, primary stimuli may also include an influence applied to the cell, such as, for example, an alteration in the growth conditions or external environment of the cells, for example, altered light intensity, temperature, osmolarity, CO₂ levels, etc; a change in the growth medium of the cells e.g. serum levels; a stress; or a change in cell cycle status.

In some embodiments of the primary screen of the invention, a further stimulus may be used in conjunction with the primary stimulus in order to delineate the arena of function of the product of a gene sequence. For example, a further stimulus might include an inhibitor (specific or otherwise) of various intracellular signalling pathways, or their components.

Imaging

In the primary screen of the present invention, the sub-cellular location of the detectable tag is observed before and after application of the stimulus in order to determine the effect of the applied stimulus. In order to improve the imaging operation, it may be desirable to introduce known markers of intracellular compartments, as discussed in more detail below in connection with the “high-throughput” section of this specification.

Techniques for the detection of a detectable tag involving the use of fluorescence detection include light microscopy, confocal microscopy and non-microscopy based fluorimetry. Preferably, the technique used will have sufficiently high resolution to determine the sub-cellular localisation of the tagged nucleotide sequence with respect to the major organelles of the cell eg. nucleus, plasma membrane, cytoplasm, golgi apparatus, mitochondria, endoplasmic reticulum etc. Sometimes however, such high resolution may not be required, for example, when trying to detect a tagged expressed nucleotide sequence being revealed on the extracellular surface of the cell. In such a case a signal (be it fluorescent or otherwise) need only be detected if the tag is exposed at the extracellular surface. Such an assay may use lower resolution microscopy, fluorimetry, ELISA or another such assay appropriate for the type of tag being used. Such methods of extracellular detection of an exposed tag are readily apparent to one skilled in the art.

Normally, after transfection and a suitable incubation period, the cells will be treated for the observation of the detectable tag produced by the transfected expression clone in question. This may involve the steps of fixation, permeabilisation, addition of antibodies etc. depending on the tag in question. Such procedures will be well known to the skilled person. Where the location of the tag is to be observed after application of the stimulus (or stimuli), the stimulus should be applied before the imaging step, and a suitable period of incubation allowed prior to observation. Thereafter, the cells may be imaged using a system appropriate for the type of tag to be detected.

The imaging may be conducted whilst the cells are live. Thus, the effect of the stimulus on live cells in real time may be observed. In this case live cells, expressing the nucleotide sequence of interest, are mounted on the microscope stage, using suitable apparatus. The cells may then be observed or imaged and the stimulus applied during this observation. Any response can then be observed or imaged in real-time as it happens. This approach is only possible with detectable tags that are themselves fluorescent such as GFP, or with probes to detect the tag that are able to enter an intact cell. It would not be possible with a non-fluorescent tag that requires detection by a fluorescent probe such as an antibody.

In the case of a GFP tagged expression clone, a typical sequence of events may be:
1. addition of the stimulus (and any further stimulus if required—this may also require a pre-incubation).
2. Fixation of the cells after an appropriate amount of time has elapsed for any response to the primary stimulus.
3. Imaging of the cells on a standard light microscope or confocal microscope.

An alternative approach, using live imaging may also be carried out as described above, in which case the sequence of events would start with step (3) and the stimulus added during observation or imaging.

High Throughput Primary Screening

Although the primary assay of the present invention may be carried out on a single nucleotide sequence, preferably, the assay is carried out in parallel, or in parallel or sequential batches, on a series of different nucleotide sequences using a given stimulus. The same series of nucleotide sequences could then be screened against a plurality of different stimuli in this way. Preferably, such a procedure would be automated to allow rapid processing of a large number of nucleotide sequences and different stimuli, if necessary. Such procedures would typically make use of multi-well plates, for example one having 96 or 384 separate wells.

Thus, for example, the primary screen of the present invention may be carried out in parallel in respect of at least 50 different nucleotide sequences using a given stimulus. Through suitable automation, the primary screen may be carried out in parallel or in sequential batches of parallel screens in respect of at least 500 nucleotide sequences and even for thousands of such sequences over relatively short periods of time (a few days), thereby allowing large scale functional analysis of expressed peptide sequences.

Where the primary screen of the present invention is to be carried out at high-throughput, the following considerations may apply.

High-Throughput Cloning and Transfection

A cDNA library, for use in the primary screen of the present invention, may for example be generated by subcloning an existing library into a vector containing a detectable tag (e.g. from Stratagene), or by generating a library in a vector containing a detectable tag, using standard techniques which will be familiar to one skilled in the art. In some circumstances, libraries of well characterised cDNA sequences in mammalian expression vectors may be available commercially. Where these libraries use a vector that is designed for recombinatorial transfer of inserts (available from eg. Invitrogen), it would then be possible to introduce these sequences into a detectable tag vector by a simple recombination step.

For example, where cloning of the nucleotide sequences into an expression vector is to be carried out on a large scale, this may be done using a high capacity cloning system such as the commercially available Gateway® cloning system from Invitrogen (Walhout et al (2000), Science 287, 116-122). This system allows the cloning of amplified nucleotide sequences such as open reading frames (ORFs) by recombination, thereby circumventing the need for screening for restriction sites that are present within the cloning sites of the cloning vector but absent from the nucleotide sequence itself. The cloned nucleotide sequence is amplified and cloned into an expression vector by homologous recombination. It is important that the sequences are cloned in a manner that allows the correct final reading frame to be achieved. This is straightforward using expression vector cloning strategies available in the art and understood by the skilled person. Another known system for high speed recombination cloning is the “Creator” system (Clontech Inc.).

These cloning systems, in combination with robotic handling systems, such as those sold by companies including Tecan, Xymark, Robbins (sold under the Hydra brand-name), make it possible to clone numerous nucleotide sequences very rapidly and with little requirement of manpower.

The step of transfecting a cell population with the expression vector containing a nucleotide sequence of interest and a sequence encoding a detectable tag may be carried out using automated equipment capable of large scale transfections. Such equipment is readily available and will known to the skilled person, again from companies such as Tecan, Xymark and Robbins.

High-Throughput Imaging

In order to effect the observation or imaging step at high-throughput, each screen may be carried out in the well of a multi-well plate (such as a 96 or 384 well plate). Microscope technology is commercially available now which permits the rapid imaging of cells in such formats, e.g. Arrayscan (from Cellomics Inc.), as well as systems available from Amersham Bioscience and Accumen. For screens that do not require microscopy to localise the tagged expressed nucleotide, there are well-established, multi-well plate reading technologies for screens with fluorimetric and colourimetric readouts.

Analysis and compilation of screening data may be automated. Suitable computer software may be written to enable the automatic detection of the subcellular localisation of a tagged expressed nucleotide sequence. Such computer software would generally work by making a comparison between the localisation of the tagged expressed nucleotide sequence and that of one or more ‘markers’ that label specific subcellular locations e.g. the nucleus, the plasma membrane, the cytosol, the golgi, the E.R etc. Such ‘markers’ are readily available commercially, for example from Molecular Probes. The following table provides examples of suitable markers which may be used.

| | Fluorescent | Compartment | requires cell |
|---|---|---|---|
| Well | label used | identified | permeabilisation? |
| 1 | Hoechst and | Nucleus and | No |
| | Transferrin- | endosomes | |
| | Alexa594 | respectively | |
| 2 | Tetramethyl | Mitochondria | No |
| | rhodamine ethyl | | |
| | ester | | |
| 3 | LysoTracker | Lysosomes | No |
| 4 | BODIPY TR | Trans-Golgi network | No |
| | ceramide | | |
| 5 | Sulfo-NHS- | Plasma membrane | No |
| | biotin/avidin | | |
| | Texas Red | | |
| 6 | Rhodamine- | Polymerised actin | Yes |
| | phalloidin | microfilaments | |

Suitable algorithms for detecting the co-localisation of a tagged expressed nucleotide sequence with several different subcellular localisations may be written. Such algorithms have already been shown to work effectively, for example the Cellomics Cytoplasm to—Nucleus Application, which is proprietary software developed by Cellomics Inc. for analysing translocation events between the cytoplasm and nucleus of a cell. With the automatic detection of the location of the product of a tagged expressed nucleotide sequence and consequently also any change in its localisation, it is possible to rapidly ‘read’ many different screening conditions in multi-well plate format.

Image analysis software that does not work on the basis of co-localisation with markers may also be employed. Thus, software may be designed that looks at the signal distribution and intensity distribution from the tagged expressed nucleotide sequence and is able to detect translocation events using this information alone.

B. Compound Library Screens

As already discussed above, the primary assay or screen of the invention may be used to identify nucleotide sequences which code for a product whose sub-cellular location is affected in the presence of a particular stimulus. A clone identified in this way can be then used in a further assay or screens in which the effect of a series of candidate drugs (typically contained in a compound library), in the presence of the agent/influence used in the primary assay, can be investigated. Through such further assays, “hit” compounds or agents can be identified which affect translocation or sub-cellular localisation of the expressed product of the clone in the presence of the agent/influence used in the primary assay.

Thus, in accordance with a second aspect of the present invention, there is provided an assay for screening a compound to determine if the compound affects the previously established capacity of an expressed tagged product to translocate within a cell of a cell population under the influence of an applied stimulus, said assay comprising:
providing a transfected cell population in which the cells contain a cloned expression vector comprising a tagged nucleotide sequence which encodes said expressed tagged product which has previously been determined to translocate under the influence of an applied stimulus;
applying the said compound to the cell population in the presence of the said stimulus; and
observing whether the presence of the compound affects translocation of the said expressed tagged product within the cell.

Where the compound is found to affect translocation of the said expressed tagged product within the cell, this may indicate that the compound interferes at a point along a pathway of signal transduction running from the point at which the stimulus impinges to the point on the pathway at which the expressed product lies, and thus may be a candidate compound for further investigation.

In this aspect of the invention, it may be desirable to determine the effect of compounds on distinctively localised but non-translocating protein as well as the effect on proteins that do translocate. This enables the user to screen for the effect of compounds on proteins that do not translocate but are distinctively localised. Thus, rather than looking for the effect of the compound on the translocation of the protein, the user may look for an effect of the compound on the distinctive localisation of the protein.

This assay of the invention is of particular advantage since the compounds are tested against targets in living cells and therefore must be able to enter the cell if they are to elicit a response. Furthermore, the tested compounds will confront the full complexity of proteins and other complex molecules present within living cells, and any toxic effects of the tested compound will be evident from defects in the growth, morphology, vitality, etc. of the cells. The assay will thus allow the identification of compounds which can be further screened for use as active pharmaceutical substances. In particular, the compounds may be active pharmaceutical substances for the treatment of a medical condition or disease associated with the expressed product of the nucleotide sequence used in the assay.

In this assay of the present invention, the capacity of the expressed product to translocate under the influence of the said applied stimulus may have been established using the primary assay of the present invention as described in detail above.

The manner and timing of the application of the stimulus, and the approach used for imaging the cells in this assay may be as described above for the primary screen of the present invention. It is most likely to be the case that compounds to be screened would be added prior to the stimulus rather than with it. This is because most compounds require some time in which to permeate the cell and have an effect. A preincubation of the cells with the compound of between minutes and hours before the stimulus is to be applied would be usual. In some circumstances, it may be possible to apply compounds at the same time or after the stimulus; this is only likely to be possible, however, where the stimulus itself is long-term/slow such as a change in the environment of the cell.

The assay may be carried out a plurality of times using the same nucleotide sequence and stimulus, but with a different compound each time. Preferably, the assay is carried out a plurality of times in parallel in a manner analogous with the high-throughput operation of the primary assay of the present invention. In this way, the assay may be used for high throughput screening of a large number of compounds.

The compound or compounds used in the assay of this aspect of the invention may comprise part of a compound library. Compound libraries are available commercially from various suppliers (e.g. Asinex (Russia) SPECS (Holland), Camgenix (USA), Contact Services (USA)). The compounds are available as either compound collections or individual compounds and the compound choice may, if desired, be based on defined chemical properties and/or structures.

C. Secondary Screens

One of the most important stages of validating a potential drug lead is determining its specificity. Once a “hit” compound or agent is identified that has the desired effect in a primary screen against a ‘target’ expressed nucleotide sequence, it is then tested to see what effect it has on other expressed nucleotide sequences. It is expected that if a compound were to effect the function of other expressed nucleotide sequences, they would most likely be those which resembled in function the original ‘target’ expressed nucleotide sequence. The effect of the “hit” compound may therefore be tested in further “secondary” screens against expressed nucleotide sequences whose products are known, or expected, to resemble, in function, that of the original ‘target’ expressed nucleotide sequence. These may be identified by assessing several criteria, for example:
1. nucleotide sequence homology with the original ‘target’ expressed nucleotide sequence;
2.protein sequence or structural homology with the product of the ‘target’ expressed nucleotide sequence;
3. known functional similarities with the ‘target’ expressed nucleotide sequence e.g. binding to the same ligands;
4. functional similarities identified as described above.

Secondary screens may also encompass a wider range of expressed nucleotide sequences whose function is not necessarily associated with, or similar to, that of the original ‘target’, or whose function is unknown.

In particular, the secondary screen of the present invention may comprise the following steps:
(a) identifying a “hit” compound in accordance with the second aspect of the present invention, where the “hit” compound is a compound which is found to alter the response of the expressed product of the target nucleotide sequence to the stimulus;
(b) transfecting a cell population with a cloned expression vector comprising a composite nucleotide sequence encoding a different nucleotide sequence to the target nucleotide sequence and a detectable tag;
(c) applying the said hit compound to the transfected cell population in the presence of the said stimulus; and
(d) applying the said stimulus to at least a portion of the transfected cell population;
(e) observing the sub-cellular location of the detectable tag in the transfected cell population after application of the stimulus and the hit compound, and comparing the observed sub-cellular localisation to the sub-cellular localisation of the detectable tag in the cell population in the presence of the stimulus but not the hit compound.

In this aspect of the invention, it is most likely to be the case that compounds to be screened would be added prior to the stimulus rather than with it. This is because most compounds require some time in which to permeate the cell and have an effect. A preincubation of the cells with the compound of between minutes and hours before the stimulus is to be applied would be usual. In some circumstances, however, it may be possible to apply compounds at the same time or after the stimulus; this is only likely to be possible where the stimulus itself is long-term/slow such as a change in the environment of the cell.

Thus, the cell population may be imaged, as already described in detail above, to determine whether the hit compound has any effect on the intracellular localization of the expressed tagged product. Such an effect may be, for example, an inhibition or enhancement, in the presence of the hit compound, of the change in localization that was originally observed in the absence of the hit compound, or a change in the intracellular site to which the expressed protein localises.

Operation of the secondary screening aspect of the present invention is analogous to the primary assay as described in detail above.

The secondary screening method of the invention may be carried out in parallel on a series of different nucleotide sequences (but with the same basic stimulus and hit compound) at high throughput in the manner which has been described above. This will enable the hit compound to be tested rapidly against a series of other expressed nucleotide sequences, including those whose function is associated with, or similar to the original target nucleotide sequence.

Alternative Assays

The assays and screens described above (the primary screen, compound screen and secondary screen) each include a step in which a cell population is transfected with a cloned expression vector which comprises a composite nucleotide sequence encoding a nucleotide sequence under investigation and a detectable tag. The changes in the way the expressed tagged product localises in the cell under a given stimulus are then observed. As an alternative to this approach, the cell population may be directly transfected with the protein of interest, tagged with a detectable tag. Thus, the tagged protein is synthesised outside the cell population and then introduced into the cells in a manner which is known per se. Thus, for example, the tagged protein may be produced in another cell type eg. bacteria or insect cells, or even synthetically before the tagged protein is extracted and then transfected into the cell in which it is to be studied. In accordance with this alternative approach, the protein of interest is amenable to the assays that are described in detail above. Methods of transfection of the tagged protein into the cell are essentially the same as those used for transfecting a nucleotide sequence into cells. Examples of commercial transfection reagents that may be used are immunofect (Immunoporation ltd.), ProVectin (Imgenex) and Chariot (ActiveMotif).

In this alternative approach, the primary assay of the present invention is an assay to determine the effect of an applied agent or influence or other stimulus on the subcellular localisation of the expressed product of a nucleotide sequence (“the nucleotide sequence under investigation”), comprising:
(i) transfecting a cell population with the expressed product of the said nucleotide sequence which expressed product includes a detectable tag;
(ii) applying the applied agent, influence or other stimulus to at least a portion of the transfected cell population;
(iii) observing the sub-cellular location of the detectable tag in the transfected cell population and comparing the observed sub-cellular localisation to the sub-cellular localisation of the detectable tag in the cell population in the absence of said applied agent or influence.

In this alternative approach, the compound screen of the invention is defined as follows:

An assay for screening a compound or agent to determine if it affects the previously established response of the product of an expressed nucleotide sequence to an applied stimulus, comprising:
i) transfecting a cell population with the expressed product of the nucleotide sequence which expressed product includes a detectable tag;
ii) applying said stimulus in the presence of said compound or agent to at least a portion of the transfected cell population; and
iv) observing whether the presence of said compound or agent affects translocation or subcellular localisation of the said expressed tagged product within the cell;
wherein the capacity of the expressed product to translocate under the influence of the said applied stimulus has been established using an assay as described above.

1. An assay to determine the effect of an applied agent or influence or other stimulus on the subcellular localisation of the expressed product of a nucleotide sequence (“the nucleotide sequence under investigation”), comprising:
(i) transfecting a cell population with a cloned expression vector comprising a composite nucleotide sequence encoding the nucleotide sequence under investigation and a detectable tag, arranged such that the product of the expressed composite nucleotide sequence includes the expressed detectable tag;
(ii) applying the applied agent, influence or other stimulus to at least a portion of the transfected cell population;
(iii) observing the sub-cellular location of the detectable tag in the transfected cell population and comparing the observed sub-cellular localisation to the sub-cellular localisation of the detectable tag in the cell population in the absence of said applied agent or influence.

2. An assay according to claim 1 which is carried out a plurality of times using a group of nucleotide sequences, each time using the same stimulus but a different nucleotide sequence from the group of nucleotide sequences, to identify nucleotide sequence (s) which encode a product whose sub-cellular localization is affected by the stimulus.

3. An assay according to claim 1, which is carried out a first plurality of times using a group of nucleotide sequences, each time using a first stimulus and respective different nucleotide sequences from the group of nucleotide sequences, in order to identify nucleotide sequence (s) which encode a product whose sub-cellular localization is affected by the first stimulus; and which is repeated a second plurality of times using the said group of nucleotide sequences, each time using a second stimulus different from the first stimulus, and respective different nucleotide sequences from the group of nucleotide sequences, to identify nucleotide sequence (s) which encode a product whose sub-cellular localization is affected by the second stimulus.

4. An assay according to claim 3 in which the assay is repeated one or more further plurality of times using different stimuli.

5. An assay according to claim 1, which is carried out a plurality of times using a different stimulus each time, but the same nucleotide sequence, to identify a stimulus or stimuli having an effect on the sub-cellular localization of the expressed product of the nucleotide sequence.

6. An assay according to claim 2, wherein at least some of the repetitions of the assay are carried out in parallel.

7. An assay for screening a compound or agent to determine if it affects the previously established response of the product of an expressed nucleotide sequence to an applied stimulus, comprising:
i) transfecting a cell population with a cloned expression vector comprising a composite nucleotide sequence encoding said nucleotide sequence and a detectable tag, arranged such that the product of the expressed composite nucleotide sequence includes the expressed detectable tag;
ii) applying said stimulus in the presence of said compound or agent to at least a portion of the transfected cell population; and
iv) observing whether the presence of said compound or agent affects translocation or subcellular localisation of the said expressed tagged product within the cell;
wherein the capacity of the expressed product to translocate under the influence of the said applied stimulus has been established using an assay as claimed in claim 1.

8. An assay according to claim 7 wherein the assay is carried out a plurality of times, using a different compound each time, and further including the step of identifying a compound or compounds which affect the sub-cellular localization of the expressed product of the nucleotide sequence in response to the stimulus.

9. An assay according to claim 8, wherein at least some of the assays, using different compounds, are carried out in parallel.

10. An assay according to claim 8 wherein the or each compound comprises part of a compound library.

11. A method of obtaining a compound which comprises (a) identifying a compound in accordance with the assay of claim 8, and (b) synthesising the compound.

12. A compound identified by the assay of claim 8.

13. A screening assay in which the compound identified in claim 8 is screened against a different expressed nucleotide sequence using the stimulus employed in the assay for screening a compound or agent.

14. A screening assay according to claim 13, wherein the compound is screened against a plurality of different expressed nucleotide sequences using the stimulus employed in the assay for screening a compound or agent.

15. A screening assay according to claim 14, wherein at least some of the individual assays are carried out in parallel.

16. An assay according to claim 1, wherein the agent, influence or other stimulus is applied to the cell population or portion thereof in an amount, and under conditions, capable of eliciting an expected response from the cells.

17. An assay according to claim 17, wherein the agent, influence or other stimulus and the expected response are selected to enable the expressed product to be identified as potentially associated with one or more aspects of cellular function or dysfunction relevant to human, animal or plant health.

18. A modification of an assay according to claim 1, wherein the cell transfection (i) is carried out using a protein tagged with a detectable tag, the said tagged protein being the pre-formed expressed product of q composite nucleotide sequence encoding the nucleotide sequence under investigation and the detectable tag.
